(19) United States
(12) Patent Application Publication
(10) Pub. No.: US 2020/0051699 A1
(43) Pub. Date: Feb. 13, 2020

(54) SYSTEM AND METHOD TO DETERMINE RELEVANT PRIOR RADIOLOGY STUDIES USING PACS LOG FILES

(71) Applicant: KONINKLIJKE PHILIPS N.V., EINDHOVEN (NL)

(72) Inventor: Amir Mohammad TAHMASEBI MARAGHOOSH, Arlington, MA (US)

(21) Appl. No.: 16/339,529

(22) PCT Filed: Oct. 9, 2017
(86) PCT No.: PCT/EP2017/075586

§ 371 Date: Apr. 4, 2019

Related U.S. Application Data
(60) Provisional application No. 62/408,086, filed on Oct. 14, 2016.

(51) Int. Cl. G16H 70/20; G16H 30/20; G16H 10/60

(52) U.S. Cl. CPC G16H 70/20; G16H 10/60; G16H 30/20

(57) ABSTRACT

A radiology workstation (10) includes a computer (12) connected to access radiology studies stored in an radiology studies archive (20) with at least one processor (22) programmed to operate the computer to: provide a user interface (24) for performing readings of radiology studies including: displaying images on a display (14) of a current radiology study being read; receiving user inputs via one or more user input devices (16) and operating on the user inputs to manipulate the display of images and to open and view past radiology studies during the reading and to receive a radiology report summarizing the reading and store the radiology report in the radiology studies archive; and recording a activity log of user inputs received via the one or more user input devices during readings of radiology studies. While providing the user interface for performing a reading by a radiologist of a current radiology study of a patient, tire at least one processor is Anther programmed to perform a relevant past radiology study recommendation process including: identifying at least one previously-read radiology study of the patient stored in the radiology studies archive as being relevant to the current radiology study of the patient using a radiologist-specific relevance identification criterion derived from content of the activity log recording the radiologist opening and viewing past radiology studies during readings performed by the radiologist; and displaying an indication of the at least one relevant previously-examined radiology study on the display.

### FIELD

The following relates to the radiology arts, radiology reporting arts, medical radiology arts, and related arts.

### BACKGROUND

Radiologists are highly specialized medical professionals. Radiologists typically review radiology studies using a Picture Archiving and Communication System (“PACS”) and/or imaging system workstation. Typically, a radiologist is expected to maintain a high workflow throughput, with the expectation that the radiologist will read a complete radiology study in a few minutes, with some more complex studies requiring more time. While efficiency is desired, each radiology reading should be comprehensive and accurate. In particular, the radiologist is expected to make reference to prior radiology readings for the imaging subject, where such readings are available and relevant.

During a typical radiology reading session, while browsing through a current radiology study, a radiologist may navigate through prior radiology studies of the same patient. This could be due to several reasons: 1) the current radiology study does not provide sufficient resolution for visualization of the targeted finding(s); 2) the current radiology study does not cover the anatomy/finding of interest or the field-of-view partially shows the finding of interest (e.g., incidental findings); 3) the radiologist is interested in comparing an observation made in the current radiology study with prior relevant radiology studies to gather comparative, differential and/or complementary information; or so forth. For example, while the radiologist is reading a chest CT image, the radiologist may also want to look at prior chest CT images (if available) to look at the growth of a nodule in lung, or may want to look at a prior PET image of the same subject to look at the functional response to a treatment.

Each radiologist may have their own approach of determining the most relevant prior radiology studies. These preferences depend upon numerous individual factors. For example, a radiologist who has a lot of experience in reading MRI imaging studies may tend to refer back to a prior MRI study (if available) when reading a CT or other non-MRI imaging study; whereas a radiologist with less experience in reading MRIs may be unlikely to refer back to a prior MRI study unless it is closely relevant. Similarly, when reading a radiology imaging study of one body part, one radiologist may find prior studies of a neighboring body part to provide a useful alternative view, whereas another radiologist may find this other study to be of little value. In practice, most radiology reading tools provide little or no assistance in identifying relevant prior radiology studies. For example, a tool might identify possibly relevant prior studies as those imaging the same body part and using the same imaging modality, mainly from DICOM meta-data (within the DICOM header). If for any reason such as human error, such information is inaccurate or unavailable, the outcome of the relevant prior determination would be invalid. A very dramatic drawback of such problem would be that the radiologist may order another radiology study, which might be unnecessary as similar radiology study may have been acquired from patient in the past. Such inefficiencies dramatically affect the radiology workflow in terms of time spent reading each case and the accuracy of diagnosis (see, e.g., Doshi, et al., “Strategies for Avoiding Recommendations for Additional Imaging Through a Comprehensive Comparison With Prior Studies”, J Am Coll Radiol 2015; 12:657-663.).

The following provides new and improved devices and methods which overcome the foregoing problems and others.

### BRIEF SUMMARY

In accordance with one aspect, a radiology workstation includes a computer connected to access radiology studies stored in a radiology studies archive. The computer includes a display, one or more user input devices, and at least one processor. The at least one process is programmed to operate the computer to: provide a user interface for performing readings of radiology studies including: displaying images on the display of a current radiology study being read; receiving user inputs via the one or more user input devices and operating on the user inputs to manipulate the display of images and to open and view past radiology studies during the reading and to receive a radiology report summarizing the reading and store the radiology report in the radiology studies archive; and recording a activity log of user inputs received via the one or more user input devices during readings of radiology studies; and while providing the user interface for performing a reading by a radiologist of a current radiology study of a patient, performing a relevant past radiology study recommendation process including: identifying at least one previously-read radiology study of the patient stored in the radiology studies archive as being relevant to the current radiology study of the patient using a radiologist-specific relevance identification criterion derived from content of the activity log recording the radiologist opening and viewing past radiology studies during readings performed by the radiologist; and displaying an indication of the at least one relevant previously-examined radiology study on the display.

In accordance with another aspect, a non-transitory computer readable medium carrying software to control at least one processor to perform an image acquisition method is provided. The method includes: providing a user interface for performing readings of radiology studies including: displaying images on the display of a current radiology study being read; receiving user inputs via the one or more user input devices and operating on the user inputs to manipulate the display of images and to open and view past radiology studies during the reading and to receive an radiology report summarizing the reading and stoic the radiology report in the radiology studies archive; and recording a activity log of user inputs received via the one or more user input devices during readings of radiology studies; and while providing the user interface for performing a reading by a radiologist of a current radiology study of a patient, performing a relevant past radiology study recommendation process including: identifying at least one previously-read radiology study of the patient stored in the radiology studies archive as being relevant to the current radiology study of the patient based on content of the activity log recording the radiologist opening and viewing past radiology studies during readings performed by the radiologist; and displaying an indication of the at least one relevant previously-examined radiology study on the display.

In accordance with another aspect, a radiology workstation includes a computer connected to access radiology studies stored in a radiology studies archive. The computer includes a display, one or more user input devices, and at least one processor. The at least one processor is programmed to operate the computer to: provide a user interface for performing readings of radiology studies including: displaying images on the display of a current radiology study being read; receiving user inputs via the one or more user input devices and operating on the user inputs to manipulate the display of images and to open and view past radiology studies during the reading and to receive an radiology report summarizing the reading and stoic the radiology report in the radiology studies archive; and recording a activity log of user inputs received via the one or more user input devices during readings of radiology studies; and while providing the user interface for performing a reading by a radiologist of a current radiology study of a patient, performing a relevant past radiology study recommendation process including: identifying at least one previously-read radiology study of the patient stored in the radiology studies archive as being relevant to the current radiology study of the patient using a radiologist-specific relevance identification criterion derived from content of the activity log recording the radiologist opening and viewing past radiology studies during readings performed by the radiologist, the content including a set of features including at least three of: image modality, body part, body section, cardinal body structure, reason for examination, procedure description, and examination date; and displaying an indication of the at least one relevant previously-examined radiology study on the display.

One advantage resides in providing a more efficient radiology workstation.

Another advantage resides in reduced bandwidth requirements for a radiology workstation.

Another advantage resides in providing a radiology workstation with faster operation.

Another advantage resides in providing a radiology workstation with an improved user interface.

Another advantage resides in extracting relevant features to determine a relevance between multiple radiology studies.

Another advantage resides in providing a user with access to more radiology studies of a single patient.

Another advantage resides in providing a model that is updated to a user's search preferences.

Further advantages of the present disclosure will be appreciated to those of ordinary skill in the art upon reading and understand the following detailed description. It will be appreciated that any given embodiment may achieve none, one, more, or all of the foregoing advantages and/or may achieve other advantages.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 shows a radiology workstation.

FIG. 2 shows a flowchart showing an exemplary method of implementing the relevant prior radiology study identification performed by the radiology workstation of FIG. 1.

FIG. 3 shows an example operation of the device of FIG. 1.

FIG. 4 shows another example operation of the device of FIG. 1.

FIG. 5 shows another example operation of the device of FIG. 1.

### DETAILED DESCRIPTION

The following is generally directed to an improved radiology reading environment, which could for example be incorporated into the Philips Intellispace PACS integrated image and information workflow system, or more generally into any radiology workstation having appropriate hardware (e.g. high resolution display, electronic data network connection with a PACS or other radiology studies archive, et cetera) for performing radiology study readings. When performing a radiology reading, the radiologist typically reviews prior radiology studies of the same patient, if such are available and deemed relevant by the radiologist. Despite following some general rules (i.e., anatomy and modality exact match), the choice of “relevant” prior radiology study varies from radiologist to radiologist. Retrieving a radiology study for review is bandwidth-intensive as transmitting the high resolution radiology images over the electronic data connection with the PACS or other radiology studies archive uses substantial bandwidth. In addition to increasing data load on the network, retrieving prior imaging studies slows down operation of the radiology workstation. Improvements to the physical operation of the radiology workstation (e.g. reduced bandwidth, faster execution) are therefore achievable by better-targeted identification and retrieval of relevant prior studies.

The following discloses improvements in which relevant prior radiology studies of a medical subject may be retrieved in a targeted manner. The disclosed approaches leverage the activity log feature of some existing radiology workstations that (if activated) tracks activities of the radiologist during radiology readings such as opening/closing imaging studies, mouse/keyboard interaction, duration of interaction, etc. The activity log is analyzed to determine the individual radiologist's preferences as to which prior radiology studies are (most) relevant, and this information is used during a current radiology reading to propose (most) relevant prior studies for review. In some embodiments, the identified most relevant studies are retrieved to the radiology workstation as a background process, thereby reducing delay when the radiologist elects to open a prior radiology study. By predicting the most relevant prior studies with the predictions targeted to the specific radiologist and to specific features of the current radiology reading task, the likelihood is substantially increased that the “correct” prior radiology studies will be suggested and (in some embodiments) pre-retrieved in background retrieval processes, thus increasing the effective bandwidth and operational speed of the radiology workstation.

In an illustrative embodiment, each radiology examination (current or prior) is labeled by a few relevant features, such as: radiology modality; body part; reason for examination; procedure description; and examination date. These features are typically already stored as PACS metadata. Because radiologists generally carry a high workload and the number of relevant features in the set of features is relatively low, it can generally be assumed that the activity log data for a specific radiologist will likely contain at least one, and possibly many, previous readings for examinations having the same set of relevant features as the exam currently being read. Prior radiology examinations that were reviewed by the radiologist during these previous reading(s) are identified from the activity log data, preferably along with a relevance metric, for example based on log data such as: number of images viewed and/or total viewing time of images of the prior examination; user interaction metrics (e.g. total scrolling distance, total number of mouse clicks, total time the prior study was open, or so forth). The feature sets of these prior examinations that were (most) relevant to the previous reading(s) are compared with feature sets of prior examinations for the same patient as the current examination, and (most) relevant prior examinations of the past patient are thereby identified.

In some embodiments, adaptive learning is employed, i.e. the log data that are analyzed are updated in real-time as log data are collected. As another variant, if the number of previous readings is too low (or zero) then the number of features used for matching can be relaxed (e.g. procedure description and/or examination date may be omitted) until sufficient log data are retrieved using the relaxed feature set. This can be particularly useful when providing suggestions for a newer radiologist having a limited log history, or for a radiologist performing an unusual examination (at least “unusual” for that radiologist).

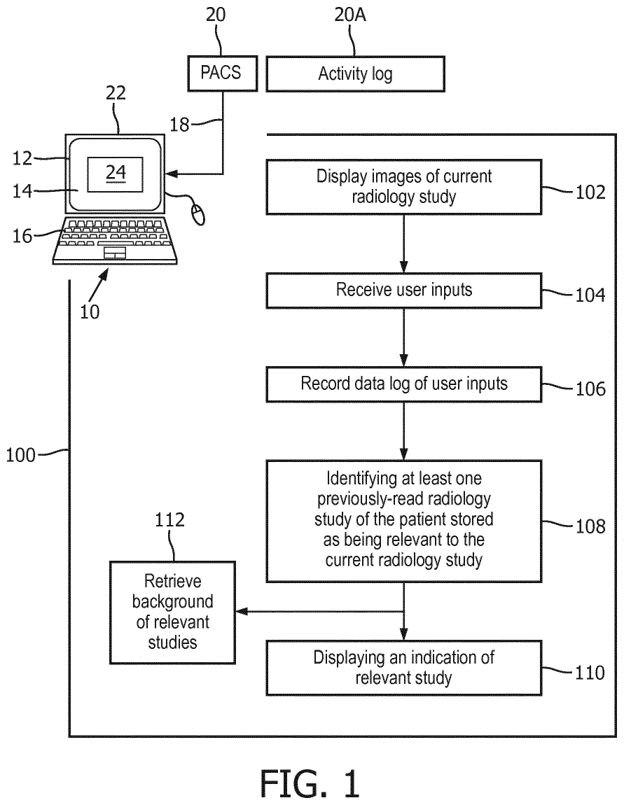

With reference to FIG. 1, an embodiment of a radiology workstation 10 is described, which may for example be implemented as a desktop computer, a “dumb” terminal connected with a network server, or any other suitable computing device to retrieve data from the server. The workstation 10 includes a computer 12 with typical components, such as at least one display component 14, at least one user input component 16, an electronic data communication link 18, an electronic database or archive 20 such as a Picture Archiving and Communication System (PACS) or any other suitable database (e.g., an electronic medical record (EMR) database), and at least one electronic processor 22 programmed to perform radiology reporting functions as disclosed herein. The at least one display 14 is configured to display one or more radiology studies, and is preferably a high resolution display in order to display high resolution radiology images. For example, a current study can be displayed on a first display, and a previously-examined radiology study, retrieved from the archive 20, can be displayed on a second display. In some examples, the display 14 can be a touch-sensitive display. The user input component 16 is configured to select at least one of the images. In some cases, the user input component 16 can be a mouse, a keyboard, a stylus, an aforementioned touch-sensitive display, and/or the like. In addition, the user input component 16 can be a microphone (i.e., to allow the user to dictate content to at least one of the radiology reports). The communication link 18 can be a wireless or wired communication link (such as a wired or wireless Ethernet link, and/or a WiFi link), e.g. a hospital network enabling the radiology workstation 10 to retrieve a stack of radiology reports making up a study from the PACS 20. In addition, the PACS or other radiology studies archive 20 is configured to store a plurality of radiology reports that include data entry fields (possibly including free-form text entry fields) by which the radiologist enters radiology findings or other observations of potential clinical significance.

The at least one processor 22 is programmed to provide a user interface via which the radiologist can display radiology images on the display 14 of a current radiology study being read, and that receives user inputs via the one or more user input devices 16 and operates on the user inputs to manipulate the display of images and to open and view past radiology studies during the reading and to receive a radiology report summarizing the reading and store the completed radiology report in the radiology studies archive 20. The radiology workstation also includes the capability of recording activity of the radiologist in an activity log 20A in the PACS 20 (as shown) or as a activity log separate from the PACS. The activity log 20A records activity of the radiologist, for example by storing keystrokes, mouse actions, or other raw user inputs, and/or storing higher-level operations performed by the radiologist such as opening a current or prior radiology examination, storing a radiology report, or so forth. The activity log 20A may be used for various purposes, such as for assessing productivity of individual radiologists and/or providing an auditable record of radiologist activities for various purposes.

The at least one processor 22 is further programmed to cause the radiology workstation 10 to perform a relevant prior radiology studies determination method, as described in more detail below, which leverages individual radiologist activity recorded in the activity log 20A to provide targeted identification of individual radiologist preferences as to which prior radiology studies are most relevant.

In some embodiments, the computer 12 is configured to receive a plurality of previous-examined radiology studies of a portion (or all) of a radiology examination subject which are stored in the PACS archive 20. These studies were previously uploaded to the PACS 20 from an imaging device (not shown) (e.g., a magnetic resonance device, an ultrasound device, a computer tomography device, a positron emission tomography device, a single-photon emission computed tomography device, and the like). In addition, the computer 12 is also configured to receive at least one “current” (i.e., not stored in the PACS archive 20) from the radiology device.

With continuing reference to FIG. 1, the at least one processor 22 of the workstation 10 is programmed to cause the workstation 10 to perform a relevant prior radiology studies determination method 100. The method 100 generally works in two operations, including (1) providing a user interface 24 for performing readings of the radiology, and (2) while providing the user interface, performing a relevant past radiology study recommendation process. The method 100 includes, while providing the user interface 24: displaying images on the display 14 of a current radiology study being read (102); receiving user inputs via the one or more user input devices 16 and operating on the user inputs to manipulate the display of images and to open and view past radiology studies during the reading and to receive an radiology report summarizing the reading and store the radiology report in the radiology studies archive (104); and recording, in the activity log 20A, activity log user inputs received via the one or more user input devices during readings of radiology studies (106). In addition, while providing the user interface for performing the reading by a radiologist of a current radiology study of a patient, the method 100 includes performing a relevant past radiology study recommendation process, which includes: identifying at least one previously-read radiology study of the patient stored in the radiology studies archive (e.g. PACS 20) as being relevant to the current radiology study of the patient using a radiologist-specific relevance identification criterion derived from content of the activity log 20A which records the radiologist opening and viewing past radiology studies during readings performed by the radiologist (108); and displaying an indication of the at least one relevant (as determined in step 108) previously-examined radiology study on the display 14 (110). Optionally, the prior studies determined in step 108 as most relevant are pre-fetched in a step 112 from the PACS or other radiology studies archive 20 as a background retrieval process performed in the background as the radiologist continues to perform the radiology reading in accord with steps 102, 104, 106.

At 102, images of a current radiology study being read by the radiologist on the display 14 of the workstation 10. For example, the at least one processor 22 is programmed to retrieve selected image(s) of the radiology study (not shown), either by receiving the images directly from an imaging device (not shown), or from the archive 20, and display it on the at least one display 14.

At 104, user inputs are received from the user input devices 16, and the user inputs are used by the at least one processor 22 to: (1) manipulate the display of images; (2) open and view past radiology studies during the reading; (3) receive a radiology report summarizing the reading; and (4) store the radiology report in the radiology studies archive 20. For example, the user may select one or more images (e.g., by clicking on it or pointing to it with the user input component 16 (i.e., a mouse), by using text entry with the user input component 16 (i.e., a keyboard), or using dictation to select the radiology report with the user input component (i.e., a microphone), or may elect to create a new report, either ab initio or, more commonly, starting from a report template. Once the report is selected or created, the report (including content entered thus far) can be displayed on the display component 14 with the image (or on a separate display component). Similarly, the user can click/type/dictate/etc. to open and view past radiology studies during the reading. In addition, the user inputs can be used to receive a radiology report that includes a summary of the study, which may, in some examples, be saved in the archive 20.

At 106, activity logs of received user inputs during radiology study readings are recorded. For example, the at least one processor 22 is programmed to collect an activity log of the user inputs, i.e., the clicks/typing/dictation on the radiology reports. Such activity log files are generated and stored during reading sessions and it captures all functionalities and user interactions with reading tool software. Examples of functionalities and interactions are opening an imaging study, and mouse and keyboard interaction in a time-stamped fashion. The activity log is then associated with the current radiology study, and stored in the PACS archive 20.

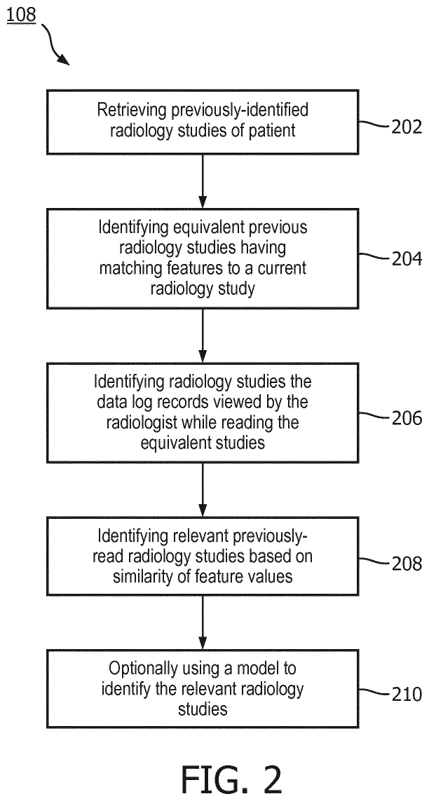

At 108, once the activity log is received/stored, at least one previously-read radiology study of the patient stored in the radiology studies archive is identified as being relevant to the current radiology study of the patient using a radiologist-specific relevance identification criterion derived from content of the activity log recording the radiologist opening and viewing past radiology studies during readings performed by the radiologist. The operations of 108 are described in more detail below, in particular in reference to FIG. 2.

At 110, an indication of the at least one relevant previously-examined radiology study is displayed the display 14. For example, a list of the previously-examined radiology studies can be displayed on the display 14, and the studies identified as relevant can be identified, e.g., by highlighting, a different font color, boding, italics, underlining, and the like. In another example, a list of only the relevant studies can be displayed.

In optional step 112, the relevant prior radiology studies are pre-retrieved to the radiology workstation 12 from the PACS 20 via the communication link 18. The pre-retrieval 112 is performed as a background process as the radiologist performs the reading of the current radiology examination in accord with operations 102, 104, 106. By “background process” it is meant that the pre-retrieval 112 is performed without intervention of (or perhaps even knowledge of) the radiologist, using free processor time and open bandwidth on the communication link 18. For example, the processor 22 performs computationally intensive activities when the user performs an operation such as resizing or otherwise manipulating a high resolution image display; however, the processor 22 may be idle, or close to idle, at other times such as when the radiologist is viewing the displayed image without actively manipulating it. These idle processor periods are suitably used to perform the pre-retrieval 112. Similarly, the pre-retrieval can be performed at time intervals when the data traffic on the communication link 18 is low, e.g. when the user is not currently retrieving an image of the current study. By action of the pre-retrieval step 112, when the radiologist operates the user input device(s) 18 to open a relevant prior radiology study it is already loaded onto the workstation 12 by the pre-retrieval 112 and hence can be opened quickly (possibly apparently instantaneously, in the view of the radiologist). By contrast, without step 112 the opening of the relevant prior radiology study is substantially delayed as the large data files storing the high resolution images are retrieved to the workstation 12 over the communication link 18. If the step 108 identifies more than one relevant prior radiology study, then the pre-retrieval can be ordered by relevance (downloading the prior study ranked of highest relevance in step 108 first), and/or the pre-retrieval can be interleaved (e.g., two or more prior studies can be pre-retrieved simultaneously as multiple downloads).

It will be appreciated that the pre-retrieval step 112 provides substantially improved effective bandwidth of the communication link 18 and substantially improves the operational efficiency of the radiology workstation 12 but only if the “correct” prior studies are identified as relevant and pre-retrieved. The step 108 synergistically provides a principled and targeted tool for identifying the prior radiology studies of the same subject as the currently read radiology study which are likely to be most relevant to the particular radiologist performing the reading in this way, the likelihood is greatly increased that the “correct” prior studies will indeed be pre-retrieved.

As previously noted, the pre-retrieval step 108 is optional. Even if it is omitted, the operational efficiency of the radiology workstation 10, and the effective bandwidth of the communication link 18, is substantially increased by providing the radiologist with the targeted list of most relevant prior studies in the display step 110. In this way, the radiologist is less likely to retrieve irrelevant or less relevant prior radiology studies, thus reducing the data load on the communication link 18 and increasing operational efficiency of the workstation 12 by eliminating wasted data transmission and time involved in retrieving and reviewing these irrelevant or less relevant prior studies.

Referring now to FIG. 2, the identification of a relevant, previously-identified radiology study of the patient (i.e., step 108 of FIG. 1) is described in more detail. The at least one processor 22 of the workstation 10 is programmed to cause the workstation 10 to perform the relevant, previously-identified radiology identification method 108. The method 108 includes: retrieving at least one previously-identified radiology study of the patient (202); identifying equivalent radiology studies read by the radiologist having feature values for a set of features that match the feature values of the current radiology study (204); identifying referenced radiology studies for which the activity log records were opened and viewed by the radiologist while reading the equivalent radiology studies (206); identifying the at least one previously-read radiology study of the patient based on similarity of feature values of the at least one previously-read radiology study of the patient to feature values of the referenced radiology studies (208); and optionally using a model to identify at least one relevant previously-reviewed radiology study (210).

At 202, metadata for at least one previously-identified radiology study of the patient is retrieved. For example, the DICOM metadata of one or more studies can be retrieved from the PACS archive 20. Additionally, the activity logs for the radiologist performing the current radiology study reading are retrieved from the PACS activity log 20A.

At 204, equivalent radiology studies read by the radiologist having feature values for a set of features that match the feature values of the current radiology study are identified. For example, the set of features can include image modality, body part, body section, cardinal body structure, reason for examination, procedure description, examination date, and/or the like. In some examples, the set of features can include at least three of these features. In other examples, the set of features can be required to include at least image modality, body part, and reason for examination. The at least one processor 22 is programmed to identify previously-identified studies as having features in common with the current study. These features are then extracted by the at least one processor 22. In another example, the image data (e.g., DICOM image data) can be processed using image-processing techniques can extract information about the study, such as modality and anatomy.

At 206, the activity log data for the readings of the equivalent previous radiology studies are processed to identify referenced radiology studies which were opened and viewed by the radiologist while reading the equivalent radiology studies. For example, the at least one processor 22 is programmed to determine all studies opened during the reading session of each equivalent previous radiology study. In a step 208, the at least one processor 22 is programmed to assign a relevance metric to each referenced radiology study based on content of the activity log relating to the opening and viewing of the referenced radiology study. In some examples, the relevance metric can include at least one criterion, such as number of images viewed; total viewing time of images of the previous examination; total scrolling distance, total number of mouse clicks, total time the prior study was open, and the like. In some examples, the relevance metric can include at least two of these criteria.

At 208, the at least one previously-read radiology study of the patient based on similarity of feature values of the at least one previously-read radiology study of the patient to feature values of the referenced radiology studies is identified. For example, the at least one processor 22 is programmed to compare the extracted features and the relevance metric of the previously-reviewed studies to the extracted features and the relevance metric of the current study. Previously-reviewed studies having the highest number of extracted features and the highest relevance metric are identified as being relevant to the current study.

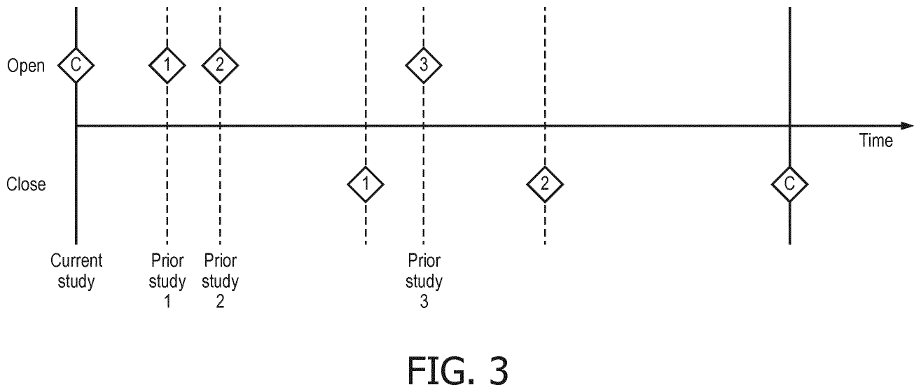

FIGS. 3-5 show several examples of how the relevant previously-reviewed studies are identified. FIG. 3 shows a scenario in which the user has opened three different imaging studies and by the end of the reading session, the user may close imaging study 1 and 2 but not 3. This is considered as a reason that study 3 is the most relevant to current study opened. Thus, the relevance metric is based on whether the study is open or closed.

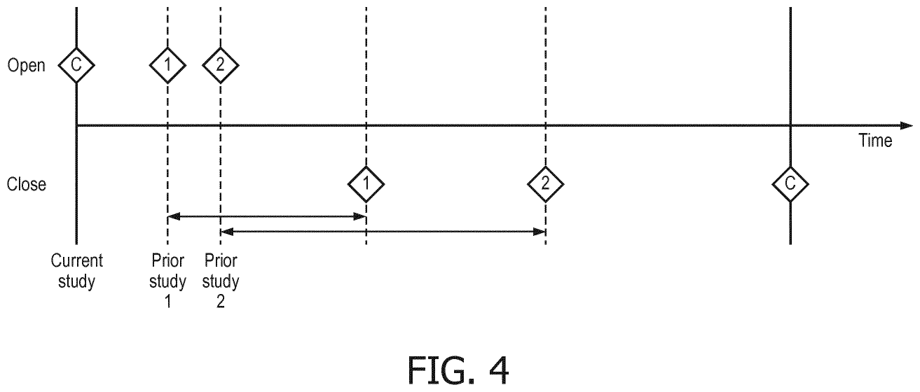

FIG. 4 shows a scenario in which the user may open two different imaging studies 1 and 2 and after a while, the user may close both images; however, as shown in the figure, imaging study stayed open longer than imaging study 1 and this is taken as an evidence that imaging study 2 is more relevant to the current opened imaging study.

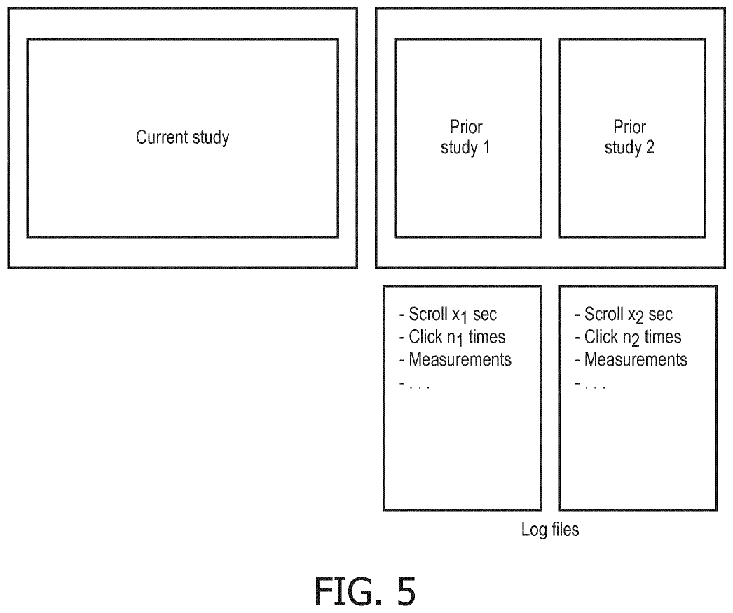

FIG. 5 shows a scenario in which the relevance is determined based on the amount of user interaction with each of the opened imaging studies. For example, the user can measure the amount of mouse scrolling, mouse clicks, etc. on each image to determine which imaging study was relevant to the current imaging.

It will be appreciated that these are only a few example of features that could be extracted from log files. Other type of features that could be indirectly linked to each imaging study opened during a reading session can also be considered. Such features can be combined together as well.

In some embodiments, at 210, once it is determined whether at least one of the previously-reviewed imaging studies is relevant to the current radiology study, a classifier can be trained to build a “relevance model” (i.e., a clustering model). To do so, the at least one processor 22 is programmed to train a model using the extracted features and the relevance metric and apply the model to the feature values of the at least one previously-read radiology study of the patient to identify relevance to the current radiology study of the patient. For example, at least a pair of imaging studies are input into the model with unique descriptors (such as modality, body part, study description, reason for examination, findings and problem list, etc.,) and based on the descriptors for a pair of imaging studies, a probability of relevance is determined between two imaging studies within a pair. It will be appreciated that different types of classification and clustering techniques can be considered. In addition, the at least one processor 22 is programmed to update the model based on the determined relevance the at least one previously-read radiology study of the patient to the current radiology study of the patient. Advantageously, the updated trained model is best fit to a profile of the current user.

Referring back to FIG. 1, the user interface 24 is now described in more detail. The user interface 24 is configured to capture, via the user input 16, a user's (reason for fetching relevant prior imaging studies, as well as interaction. The reasons for user selection can include (1) better resolution of images (e.g., CT 1 mm spacing vs. CT 0.5 mm spacing); (2), images that provide more coverage of the patient; (3) comparison of two images, (e.g., CT time point 1<->CT time point 2); and images that complement one another (e.g., CT images vs PET images of the same area of a patient).

Depending on user's selection, different criteria can be considered to fetch appropriate machine-learning methods and RPM packaging managers. Furthermore, the user can prioritize the feature(s) to be used to determine relevance based on their preference (e.g., modality, anatomy, finding, time, etc.). Furthermore, the interface 24 can be utilized so that the user can select to run the system in “learning mode” during which the system automatically adapts to user's correction to automatic outputs (i.e., during the updating process). In addition, a part of the interface 24 can be dedicated for data source selection. For example, the user can specify whether the features to be extracted from DICOM meta-data, radiology reports, EMR-based problem list, etc.

It will be appreciated that the various documents and graphical-user interface features described herein can be communicated to the various components 12, 14, 16, 18 20, and data processing components 22 via a communication network (e.g., a wireless network, a local area network, a wide area network, a personal area network, BLUETOOTH®, and the like).

The various components 12, 14, 16, 18, 20, of the workstation 10 can include at least one microprocessor 22 programmed by firmware or software to perform the disclosed operations. In some embodiments, the microprocessor 22 is integral to the various components 12, 14, 16, 18, 20, so that the data processing is directly performed by the various components 12, 14, 16, 18, 20. In other embodiments the microprocessor 22 is separate from the various components. The data processing components 22 of the workstation 10 may also be implemented as a non-transitory storage medium storing instructions readable and executable by a microprocessor (e.g. as described above) to implement the disclosed operations. The non-transitory storage medium may, for example, comprise a read-only memory (ROM), programmable read-only memory (PROM), flash memory, or other repository of firmware for the various components 12, 14, 16, 18, 20, and data processing components 22. Additionally or alternatively, the non-transitory storage medium may comprise a computer hard drive (suitable for computer-implemented embodiments), an optical disk (e.g. for installation on such a computer), a network server data storage (e.g. RAID array) from which the various component 12, 14, 16, 18, 20, data processing components 22, or a computer can download the device software or firmware via the Internet or another electronic data network, or so forth.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A radiology workstation comprising:
a computer connected to access radiology studies stored in an radiology studies archive the computer including a display one or more user input devices, and at least one processor programmed to operate the computer to:
provide a user interface for performing readings of radiology studies including:
displaying images on the display of a current radiology study being read;
receiving user inputs via the one or more user input devices and operating on the user inputs to manipulate the display of images and to open and view past radiology studies during the reading and to receive a radiology report summarizing the reading and store the radiology report in the radiology studies archive; and
recording a activity log of user inputs received via the one or more user input devices during readings of radiology studies; and
while providing the user interface for performing a reading by a radiologist of a current radiology study of a patient, performing a relevant past radiology study recommendation process including:
identifying at least one previously-read radiology study of the patient stored in the radiology studies archive as being relevant to the current radiology study of the patient using a radiologist-specific relevance identification criterion derived from content of the activity log recording previous activity data including the radiologist opening and viewing past radiology studies during readings performed by the radiologist; and
displaying an indication of the at least one relevant previously-examined radiology study on the display.

2. The radiology workstation according to claim 1, wherein the identifying comprises:
identifying equivalent radiology studies read by the radiologist having feature values for a set of features that match the feature values of the current radiology study;
identifying referenced radiology studies the activity log records were opened and viewed by the radiologist while reading the equivalent radiology studies; and
identifying the at least one previously-read radiology study of the patient based on similarity of feature values of the at least one previously-read radiology study of the patient to feature values of the referenced radiology studies.

3. The radiology workstation according to claim 2, wherein the set of features includes at least three of: image modality, body part, body section, cardinal body structure, reason for examination, procedure description, and examination date.

4. The radiology workstation according to claim 2, wherein
the set of features includes at least image modality, body part, body section, cardinal body structure, and reason for examination.

5. The radiology workstation according claim 1, wherein the identifying of referenced radiology studies further includes:
assigning a relevance metric to each referenced radiology study based on content of the activity log relating to the opening and viewing of the referenced radiology study including at least two of: number of images viewed; total viewing time of images of the previous examination; total scrolling distance, total number of mouse clicks, and total time the prior study was open.

6. The radiology workstation according to claim 1, wherein the identifying of the at least one previously-read radiology study of the patient based on similarity of feature values includes:
training a model using the extracted features and the relevance metric; and
applying the model to the feature values of the at least one previously-read radiology study of the patient to identify relevance to the current radiology study of the patient.

7. The radiology workstation according to claim 6, wherein the at least one processor is further programmed to:
update the model based on the determined relevance the at least one previously-read radiology study of the patient to the current radiology study of the patient.

8. A non-transitory computer readable medium carrying software to control at least one processor to perform an image acquisition method, the method including:
providing a user interface for performing readings of radiology studies including:
displaying images on the display of a current radiology study being read;
receiving user inputs via the one or more user input devices and operating on the user inputs to manipulate the display of images and to open and view past radiology studies during the reading and to receive a radiology report summarizing the reading and store the radiology report in the radiology studies archive; and
recording a activity log of user inputs received via the one or more user input devices during readings of radiology studies; and
while providing the user interface for performing a reading by a radiologist of a current radiology study of a patient, performing a relevant past radiology study recommendation process including:
identifying at least one previously-read radiology study of the patient stored in the radiology studies archive as being relevant to the current radiology study of the patient based on content of the activity log recording previous activity data including the radiologist opening and viewing past radiology studies during readings performed by the radiologist; and
displaying an indication of the at least one relevant previously-examined radiology study on the display.

9. The non-transitory computer readable medium according to claim 8, wherein the identifying comprises:
identifying equivalent radiology studies read by the radiologist having feature values for a set of features that match the feature values of the current radiology study;
identifying referenced radiology studies the activity log records were opened and viewed by the radiologist while reading the equivalent radiology studies; and
identifying the at least one previously-read radiology study of the patient based on similarity of feature values of the at least one previously-read radiology study of the patient to feature values of the referenced radiology studies.

10. The non-transitory computer readable medium according to claim 9, wherein the set of features includes at least three of: image modality, body part, body section, cardinal body structure, reason for examination, procedure description, and examination date.

11. The non-transitory computer readable medium according to claim 9, wherein the set of features includes at least image modality, body part, and reason for examination.

12. The non-transitory computer readable medium according to claim 9, wherein the identifying of referenced radiology studies further includes:
assigning a relevance metric to each referenced radiology study based on content of the activity log relating to the opening and viewing of the referenced radiology study including at least two of: number of images viewed; total viewing time of images of the previous examination; total scrolling distance, total number of mouse clicks, and total time the prior study was open.

13. The non-transitory computer readable medium according to claim 9, wherein the identifying of the at least one previously-read radiology study of the patient based on similarity of feature values includes:
training a model using the extracted features and the relevance metric; and
applying the model to the feature values of the at least one previously-read radiology study of the patient to identify relevance to the current radiology study of the patient.

14. The non-transitory computer readable medium according to claim 13, further including:
updating the model based on the determined relevance the at least one previously-read radiology study of the patient to the current radiology study of the patient.

15. The radiology workstation according to claim 2,
wherein the content includes a set of features including at least three of: image modality, body part, body section, cardinal body structure reason for examination, procedure description, and examination date; and
displaying an indication of the at least one relevant previously-examined radiology study on the display.

16. (canceled)

17. (canceled)

18. (canceled)

19. (canceled)

20. (canceled)
